(19) United States
(12) Patent Application Publication
(10) Pub. No.: US 2023/0152337 A1
(43) Pub. Date: May 18, 2023

(54) Analysis Device

(71) Applicant: Biosystems Assure Ltd, Poole (GB)

(72) Inventor: James McQuillan, Poole (GB)

(21) Appl. No.: 17/906,767

(22) PCT Filed: Feb. 23, 2021
(86) PCT No.: PCT/EP2021/054454

§ 371 Date: Sep. 20, 2022

(30) Foreign Application Priority Data
Mar. 20, 2020 (GB) 2004062.2

(51) Int. Cl. G01N 35/00; G01N 35/04; G01N 35/02

(52) U.S. Cl. CPC G01N 35/00009; G01N 35/04; G01N 35/025; G01N 35/00871; G01N 2035/0403; G01N 2035/00306; G01N 2035/009

(57) ABSTRACT

An analysis device is described comprising a housing 12, and a carrier 14 within the housing, the carrier 14 including a plurality of individual pockets 18, each pocket 18 containing a stable assay, and a first seal member 20 operable to cover at least one of the pockets 18 to substantially seal the said pocket(s) 18.

This invention relates to an analysis device, for example suitable for use in conducting DNA and/or RNA analysis. In particular, it relates to a device that does not require a significant supporting infrastructure and so makes it possible to perform high quantity, high-integrity biological analyses “in the field” or away from a laboratory, if desired, although the invention is not restricted to such use and may be used in other applications or environments.

Where analysis tasks are required to be undertaken, currently the analyst typically has to transport large quantities of equipment to the field to allow the analysis to be performed. The level of equipment required is such that this task can be challenging and impractical. Alternatively, samples to be analysed may be transported to a remote laboratory for analysis. The remote laboratory approach involves local sampling and then sample transportation to a central laboratory facility, and this approach introduces delays in obtaining results and cross-contamination risks into the process. The delays may result in required tests not being completed before goods are shipped, for example, and so raises the risk of unnecessary process, commercial, business or other impacts, and the cross-contamination risks reduce analysis accuracy and raise the chance of erroneous false-positive results being returned.

Where the analysis is used to test individuals for specific biological infections such as E. coli, C. difficile or coronavirus (COVID-19), the typical analysis regimes can take over 48 hours to obtain a result, during which time the risks may have spread.

There are currently no reliable analysis techniques that permit an unskilled or semi-skilled operator to sample and accurately analyse biological targets “in the field” in a short space of time. The biggest issue in conventional analysis techniques is the handling of the assay reagent and the sampling process for the target to be analysed.

There is a need for an analysis technique and an analysis device for use therein which can efficiently and accurately provide an analysis result within a short period of time. There is a need for an analysis technique which can be safely and effectively carried out by an unskilled or semi-skilled operator. One objective of the invention to provide such a device and technique, in which at least some of the disadvantages associated with known techniques and devices are overcome or are of reduced impact.

According to a first aspect of the present invention, there is provided an analysis device comprising a housing, and a carrier located within the housing, the carrier including a plurality of individual pockets, each pocket containing a stable assay reagent material, and a first seal member positionable over at least one of the pockets to substantially seal the said pocket(s).

The carrier conveniently comprises a reel-to-reel tape. Alternatively, it may comprise a rotatable disc. The first seal member conveniently comprises a tape that can be applied to the carrier to substantially seal the pockets thereof, in turn.

The assay device preferably further comprises a second seal member. The second seal member conveniently comprises a tape that can be removed from the carrier to uncover the pockets thereof, in turn.

The carrier is preferably moveable relative to the housing. The housing is preferably provided with an access window, and the carrier is preferably movable to register or align the pockets thereof, in turn, with the access window to allow target samples to be introduced into the pockets, in turn. A movable shutter is preferably provided and operable to close the access window and prevent or restrict access to the interior of the housing.

Preferably, the second seal member is removed from each pocket before, preferably immediately before, that pocket is moved into alignment with the window. In this manner, the assay reagent material within each pocket can be kept under sterile and/or other preferred storage conditions until the point at which the target sample is introduced thereto.

Preferably, the first seal member is applied to each pocket after, preferably immediately after, movement of that pocket away from the window. In this manner, the target sample introduced into the pocket is stored in a sealed manner, and so presents minimal risk to an operator or other personnel in the vicinity of the assay device.

A shield device is preferably provided and arranged to be fitted to the device, to guide the introduction of the target sample into the pocket and protect the surrounding parts of the device from contamination. Where a swab is used in the introduction of the target sample, the shield device conveniently becomes attached to the swab upon the introduction of the target sample so that the shield device is removed from the device, in use, with the swab.

According to a second aspect of the present invention, there is provided an assay testing method comprising providing an analysis device as set out hereinbefore, moving the carrier to a first position providing access to a first pocket thereof, depositing a target sample within first pocket, and moving the carrier to a second position, wherein upon movement of the carrier to the second position the first seal member is applied to close and substantially seal the first pocket.

It will be appreciated that the invention provides an apparatus which enables a stable assay reagent material to be stored in pockets within the carrier prior to exposure to a target sample. The apparatus ensures that the assay reagent material is preserved in the pockets prior to exposure to a target sample.

As set out hereinbefore a contamination shield device is preferably provided and used to aid the introduction of the target sample into the first pocket and to avoid contamination of surround parts of the device.

The sample treated assay pocket may be stored successfully for a period of time prior to analysis as the treated assay pocket is sealed from an external environment by the first seal member. During analysis, the test sample treated assay pocket is analysed to confirm the presence or absence of the target contaminant. This may be undertaken with the first seal member still in position, so exposure of a target sample need not occur.

The apparatus may be configured to carry out single or multiple tests which can be successfully stored within a single housing. By way of example, tests conducted upon a number of individuals may all be contained within a single housing.

The analysis apparatus provides a controlled mechanism for delivering a stable assay for exposure to a test sample when required. It enables effective end-to-end sampling and analysis process.

The apparatus is simple and easy to use enabling the apparatus to be handled by unskilled operators.

The apparatus, for example the housing, may further comprise a protective cover, in the form of for example a protective shutter, operable to prevent access to the interior of the housing.

The first and/or second seal member may, in one embodiment, be applied by exposure to heat, for example a heat stamp. An adhesive may be applied to a portion of the pocket, for example. The adhesive may be heat activated.

The pockets may be accessed by an operator securely and without risk of cross-contamination. The test sample may be easily introduced into the recess of the pocket and applied to the assay stored therein. The test sample may be treated according to the specific assay type and process to reveal the DNA and RNA.

An analyser unit may be provided for use in conjunction with the assay device.

The analyser unit may be configured to detect DNA and RNA within the test sample, if present, by illuminating the sample with light at the appropriate wavelength and monitoring the sample for release of light at the characteristic frequency of the contaminant being sought. The emitted light intensity is recorded for the duration of the process and then uploaded to an attached computer or smartphone where it is examined to determine whether or not the contaminant is present and, if it is, the rate of increase of its population.

The method of analysis of the present invention may further comprise use of the analyser unit for determining presence of a contaminant within a pocket.

The apparatus may further comprise a transmitter configured to transmit analysis information data to a receiver configured to receive the analysis information data and to process the received analysis information data. The receiver may be configured to relay the received analysis information data to another system for onwards communication, for example to a nearby office, security station or other facility to support the provision of a response to the analysis information data results.

The analyser unit may communicate with the smartphone or computer using standard communications techniques including Bluetooth®, Wi-Fi® or other short-range communications technique.

The apparatus may further comprise additional electronic systems configured to gather suitable data on the local environment and location and a) to retain that data in a secure repository and/or b) to relay suitable information to a third party for possible action using standard communications, web-based systems or modern Android®, Apple® or other applications (“Apps”). This data may be uploaded to the central database at the earliest opportunity to provide a detailed, searchable history of all measurements in one or multiple fields.

The analyser unit may be a biological sample processing unit.

The assay device may contain a dry-preserved assay reagent located within one or more pockets of the carrier.

The analyser unit may comprise an optical transmitter and receiver in communication with each other, in which the analyser unit is configured in use to energise and detect a biological target.

The analyser unit may further comprise a display configured in use to show the results from the transmitter and/or receiver. The analyser unit may be operable to share the result with a smartphone or computer.

The invention will further be described, by way of example, with reference to the accompanying drawings, in which:

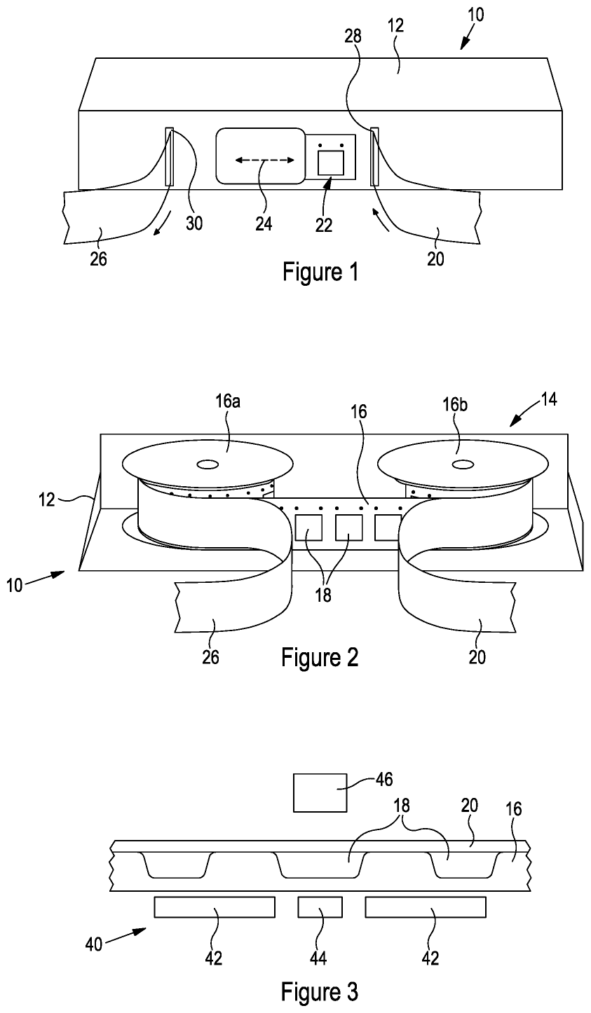

FIG. 1 is a view illustrating an analysis device in accordance with an embodiment of the invention;

FIG. 2 is a diagram illustrating parts of the analysis device;

FIG. 3 is a diagrammatic representation of an analyser unit in use in conjunction with the analysis device; and

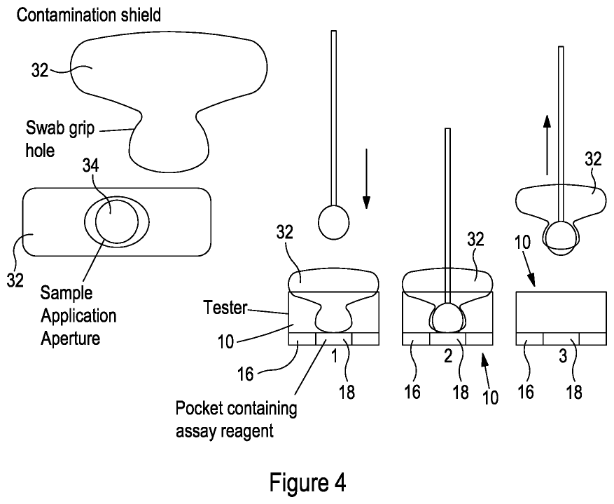

FIG. 4 is a series of views illustrating part of the device, in use.

Referring to the accompanying drawings, an analysis device 10 in accordance with an embodiment of the invention is illustrated, the device 10 comprising a housing 12 defining a cavity, and a carrier 14 located within the cavity. The carrier 14, in this embodiment, comprises a reel-to-reel tape 16, but it will be appreciated that the invention is not restricted in this regard and could take other forms. By way of example, it could be of disc-shaped form. The tape 16 is shaped to define a plurality of discrete open pockets 18, opening to a face of the tape 16, each pocket 18 comprising a recess containing a stable assay reagent material (not shown). The nature of the assay reagent material will depend upon the type of test or analysis being undertaken and is not of relevance to the present invention and so is not described herein in further detail.

The device 10 further comprises a first seal member 20 that is operable, in use, to substantially seal at least some of the pockets 18, and a second seal member 26 that, similarly, is operable, in use, to substantially seal at least some of the pockets 18. The seal members 20, 26 conveniently take the form of tapes of a suitable flexible material that can be adhered or otherwise secured to the tape 16 to close and seal the pockets 18.

The tape 16 is prepared (typically elsewhere) with the reagent material located within the pockets 18, and with the second seal member 26 affixed to the tape 16 to seal the pockets 18 with the assay reagent material located therein. Whilst the pockets 18 may all contain the same assay reagent material, this need not always be the case, and certain of the pockets 18 may include different assay reagent materials, for example to serve as controls, or to allow a series of different tests or the like to be performed.

The tape 16 is moveable relative to the housing 12 past an access window 22 formed in the housing 12 so as to align the pockets 18, in turn, with the access window 22. A shutter 24 is moveable attached to the housing 12 and is moveable between a closed position in which it closes the access window 22, and an open position.

The tape 16 is wound upon and extends between reels 16a, 16b and passes adjacent the access window 22. As illustrated, the housing 12 conveniently includes openings 28, 30 through which parts of the seal members 20, 26 project. The pockets 18 in the parts of the tape 16 wound onto the reel 16a and between the reel 16a and the opening 30 are sealed by the second seal member 26, as mentioned hereinbefore. Similarly, the pockets 18 in the parts of the tape 16 wound onto the reel 16b and between the reel 16b and the opening 28 are sealed by the first seal member 20. The pockets 18 in the part of the tape 16 between the openings 28, 30 are uncovered in the sense that they are not closed and sealed by either of the first and second seal members 20, 26. These pockets 18 are, however, contained within the housing 12, and so access thereto is restricted.

In use, when a user has a target sample ready for analysis, the shutter 24 is opened, and the user loads the target sample into the pocket 18 immediately adjacent the window 22. The shutter 24 is then closed.

As shown in FIG. 4, the step of loading the target sample into the pocket 18 conveniently includes fitting a shield device 32 into the window 22 which serves to guide the introduction of a swab or the like through the window 22 and into the pocket 18, whilst also shielding or protecting the adjacent parts of the device 10 from contamination. Upon full introduction of the swab or the like into the pocket 18, the shield device 32 conveniently becomes attached to the swab or the like, so that upon subsequent removal of the swab or the like, the shield device 32 is removed from the device 10. As illustrated, the shield device 32 is of generally funnel like form, including an opening 34 through which the swab is exposed, in use, when fully inserted, to the contents of the pocket 18.

Once the sample is received within the pocket 18, the reels 16a, 16b are rotated, for example using suitable stepper motors, to move the tape 16 to bring the next one of the pockets 18 into alignment with the window 22. During such winding, part of the seal member 20 is introduced into the housing 12 through the opening 28 to overlie the pocket 18 that newly contains the target sample, and is sealed to the tape 16 to close and seal that pocket. By way of example, this may be achieved using a heat stamp to activate a heat activated adhesive applied around the pocket 18, for example. An alternative option may be to heat weld the seal member 20 in position. It will be appreciated that other techniques may be used without departing from the scope of the invention. The contents of the pocket 18 containing the newly introduced target sample are thus substantially sealed from the external environment, reducing or avoiding the risk of contamination and so enhancing test accuracy, and also providing protection for the user and others in the vicinity of the device.

The newly sealed pocket 18 may thus be moved to a storage position within the housing 12 of the device, becoming wound upon the reel 16b.

In addition, the movement of the tape 16 allows removal, for example by unpeeling, of part of the seal member 26 from part of the tape 16, thus exposing the next one of the pockets 18 to be aligned with the window 22, ready for introduction of the next sample.

The analysis device conveniently further comprises an analyser unit (see FIG. 3). The housing 12, after having been loaded with samples to be analysed, may for example be inserted into the analyser unit 40.

The analyser unit 40 is conveniently configured to read the assay material type and reports this information to a computer program or application (referred to herein as an “App”). The App may be configured to initiate a test sequence based upon this information, the test sequence requiring inputs such as the sample and target details.

The sealed pocket 18 is moved to an analysis point to enable analysis of the contents of the pocket 18, for example along with the positive and negative control pockets.

The analyser unit 40 conveniently includes heat plates 42 adjacent which the pockets 18 are passed to heat or cool the pocket contents to a desired temperature and/or to hold the pocket contents at that temperature for a predetermined dwell time. In a simpler arrangement, a single heat plate may be used to raise the temperature of the contents of the pockets 18 to allow the performance of omnithermal tests. Subsequently, the pockets 18 are illuminated, in turn, using a light source 44 of a predetermined wavelength, and the fluorescence from the pockets is measured by a detector 46. The data derived in this manner may indicate whether target is present in the sample and, if so, the concentration of the target using the amount of light emitted at the characteristic wavelength. This illumination and fluorescence measurement may be repeated at various time points, determined by the nature of the test or analysis to be performed, to determine whether or not the target is present and, as the light output increases, the point at which it crosses thresholds.

The analyser unit 40 may include a transmitter configured to transmit the analysis information data to a remote receiver configured to receive the analysis information data, and to process the received analysis information data. The receiver may be configured to relay the received analysis information data to another system for onwards communication, for example to a local computer, nearby office or other facility to support the provision of a response to the received analysis information, if required. This may be delivered directly, for example through a computer network, or through a web server, or through an Android™- or Apple™-style smart-phone application.

In one mode of operation, therefore, the target assay analyser unit 40 may receive the analysis apparatus housing 12 and operate to recognise the assay type. The unit 40 communicates with the App and they confirm that the correct analysis process is loaded. The App then directs data entry, the sampling and sample application to the assay. Once loaded, the unit automatically processes the sample using one or more of the heat plates and stepper motors or the like to drive the reels for rotation, and communicates the results to the App.

The App configures the data and uploads this to a central database when the network is available.

The uploaded data may include user data from the App, location data from the device, along with temperature and humidity readings and other parameters, to enrich the data.

The analyser unit 40 may be deployed singly or as a networked sensing system. In this case the system may be connected to the end user in a number of ways. This includes the network interface to port data into the network for a single or multiple analyses. It also includes the application that is required at the user or owner site to receive and display relevant information and this will be in the form of an application (on a smart-phone or tablet) or a web service or a bespoke display.

Whilst a specific embodiment of the invention is described hereinbefore, it will be appreciated that a wide range of modifications and alterations may be made thereto without departing from the scope of the invention as defined by the appended claims.

1. An analysis device comprising a housing, and a carrier within the housing, the carrier including a plurality of individual pockets, each pocket containing a stable assay, and a first seal member operable to cover at least one of the pockets to substantially seal the said at least one pocket.

2. A device according to claim 1, wherein the carrier comprises a reel-to-reel tape.

3. A device according to claim 1, wherein the carrier comprises a rotatable disc.

4. A device according to claim 1, wherein the first seal member-comprises a tape that can be applied to the carrier to substantially seal at least some of the pockets thereof, in turn.

5. A device according to claim 1, further comprises a second seal member.

6. A device according to claim 5, wherein the second seal member comprises a tape that can be removed from the carrier to uncover the pockets thereof, in turn.

7. A device according to claim 1, wherein the carrier is moveable relative to the housing.

8. A device according to claim 7, wherein the housing is provided with an access window, and the carrier is movable to register or align the pockets thereof, in turn, with the access window to allow target samples to be introduced into the pockets, in turn.

9. A device according to claim 8, wherein a movable shutter is provided and operable to close the access window and prevent or restrict access to the interior of the housing.

10. A device according to claim 8, further comprising a shield device receivable within the access window to shield adjacent parts of the device from contamination.

11. A device according to claim 7 and further comprising a second seal member, comprising a configuration wherein the second seal member is removed from each pocket before that pocket is moved into alignment with the window.

12. A device according to claim 7, comprising a configuration wherein the first seal member is applied to each pocket after movement of that pocket away from the window.

13. A device according to claim 1, comprising a configuration wherein the first seal member is applied using a heat sealing or other suitable arrangement.

14. A device according to claim 1, in which the housing further comprises an analysis point, and is configured to provide the stored and sealed pocket comprising a test sample to the analysis point for subsequent analysis processing.

15. A device according to claim 1, further comprising an analyser unit operable to obtain assay information data from the or each pocket.

16. A device according to claim 15, wherein the analyser unit further comprises a transmitter operable to transmit assay information data to a remote receiver.

17. A device according to claim 15, further comprising an alarm operable for activation when the analyser unit detects the presence of at least one predetermined bioform.

18. An assay testing method comprising the steps of providing an analysis device according to claim 1, moving the carrier to a first position providing access to a first pocket thereof, depositing a target sample within first pocket, and moving the carrier to a second position, wherein upon movement of the carrier to the second position the first seal member is applied to close and substantially seal the first pocket.
